(19) United States
(12) Patent Application Publication
(10) Pub. No.: US 2015/0173607 A1
(43) Pub. Date: Jun. 25, 2015

(54) METHOD FOR ACQUIRING OPTICAL TOMOGRAPHIC IMAGE

(71) Applicant: Sumitomo Electric Industries, Ltd., Osaka-shi (JP)

(72) Inventors: Mitsuharu Hirano, Yokohama-shi (JP); Takemi Hasegawa, Yokohama-shi (JP); Ichiro Sogawa, Yokohama-shi (JP); Masato Tanaka, Yokohama-shi (JP)

(21) Appl. No.: 14/414,051

(22) PCT Filed: Jul. 11, 2013
(86) PCT No.: PCT/JP2013/069023

§ 371 Date: Jan. 9, 2015

(30) Foreign Application Priority Data
Jul. 11, 2012 (JP) 2012-155795

(51) Int. Cl. A61B 3/10

(52) U.S. Cl. CPC A61B 3/102

(57) ABSTRACT

A method for acquiring an optical tomographic image is disclosed. The method acquires the optical tomographic image based on a result of a Fourier transform of an interference light spectrum obtained by dividing light into first branched light and second branched light and causing mutual interference between reflected light and diffused reflected light. The reflected light arises at a reflecting body when the first branched light is irradiated onto the reflecting body. The diffused reflected light arises when the second branched light is irradiated onto an examination region. The acquisition method includes: acquiring a first optical tomographic image when an examination object is arranged in the examination region; acquiring a second optical tomographic image when the examination object is not arranged in the examination region; and acquiring the optical tomographic image based on a difference between the first optical tomographic image and the second optical tomographic image.

### TECHNICAL FIELD

The present invention relates to a method for acquiring an optical tomographic image.

### BACKGROUND ART

Technology for acquiring an optical tomographic image based on optical coherence tomography (OCT) can be used to measure the distribution of a reflection amount in a depth direction of an examination object using optical interference. In recent years, this technology for acquiring an optical tomographic image has been applied to bioinstrumentation since an internal structure of an examination object can be imaged with high spatial resolution.

An apparatus for acquiring an optical tomographic image based on OCT divides light output from a light source unit into first branched light and second branched light, and causes reflected light generated at a reflecting body when the reflecting body is irradiated with the first branched light, and diffused reflected light generated at an examination object when the examination object is irradiated with the second branched light to interfere with each other. The acquisition apparatus then detects the power of the interference light using a detection unit, and analyzes the detection result to obtain a reflection information distribution (one-dimensional optical tomographic image) in a depth direction of the examination object. Further, a two-dimensional or three-dimensional optical tomographic image of the examination object can be acquired by scanning a light irradiation position with respect to the examination object.

Among the OCT methods, TD-OCT (time-domain OCT) utilizes a fact that, when a light source unit which outputs light having a short coherence length is used, an amplitude of interference light decreases if there is a difference in the light path lengths between both lights from the light source unit to a detection unit, and the amplitude of the interference light increases only when there is no difference in the light path lengths between both lights from the light source unit to the detection unit. In TD-OCT, since it is possible to obtain reflection information for positions in the depth direction of an examination object in accordance with a position of a reflecting body, a distribution of the reflection information in the depth direction of the examination object can be obtained by detecting an interference light amplitude while moving the reflecting body. However, in TD-OCT, since it is necessary to move the reflecting body mechanically in order to obtain the reflection information distribution in the depth direction of the examination object, the time taken to acquire a tomographic image of the examination object is long.

On the other hand, among the OCT methods, FD-OCT

(Fourier-domain OCT) utilizes the wavelength dependence of an interference signal, and requires less time to acquire an optical tomographic image of an examination object in comparison to TD-OCT. When light that is output from a light source unit is equally divided into first branched light and second branched light, an intensity P(k) of the interference signal for the light of a wave number k is expressed by the following equation:

P(k)=P₀/4{Rₛ+Rₘ+2(RₛRₘ)^{1/2} cos (2 kz)},

where P₀ represents the power of the light output from the light source unit, k(=2π/λ) represents the wave number of the light, z represents the depth direction position of the examination object, Rₛ represents the reflectance at the examination object, and Rₘ represents the reflectance at the reflecting body.

As will be understood from the above equation, the intensity P(k) of the interference signal for the light of wave number k vibrates at a period that is in accordance with the depth direction position z of the examination object with an amplitude that is proportional to the reflectance Rₛ to the power of ½ at the examination object. Therefore, when a spectrum of the interference signal detected by the detection unit is subjected to a Fourier transform on a wave number axis 2k, a result thereof indicates the reflectance Rₛ at the depth direction position z of the examination object (that is, a reflectance distribution in the depth direction). FD-OCT utilizes this fact.

That is, in FD-OCT, when light is irradiated at an examination object, if the light penetrates as far as the inside of the examination object and diffuse reflection occurs at each position along the optical axis, an interference signal that is detected by the detection unit appears in a form in which signals for respective positions inside the examination object overlap. When a Fourier transform is performed on such an interference signal, a reflection distribution in the depth direction of the examination object is directly obtained. In FD-OCT, since it is necessary to measure a spectrum, a spectroscope is used as the detection unit. In FD-OCT, since it is not necessary to mechanically move a reflecting body, the time taken to acquire an optical tomographic image of the examination object is shorter in comparison with TD-OCT.

### CITATION LIST

### Patent Literature

Patent Literature 1: Japanese Patent Application Laid-Open Publication No. 2004-223269
Patent Literature 2: Japanese Patent Application Laid-Open Publication No. 2012-085844
Patent Literature 3: Japanese Patent Application Laid-Open Publication No. 2010-014668

### SUMMARY OF INVENTION

### Technical Problem

Although such technology for acquiring an optical tomographic image based on OCT acquires an intensity distribution of diffused reflected light generated at respective positions of an examination object as an optical tomographic image, in some cases diffused reflected light that, for reasons relating to the apparatus or the measurement, arises at a place other than the examination object also reaches the detection unit and contributes to interference. In an optical tomographic image that is acquired at such a time, a false peak that is derived from the diffused reflected light that is generated at a place other than the examination object is superimposed on a true optical tomographic image. When a false peak is superimposed on a true optical tomographic image of the examination object, a true optical tomographic image of the examination object cannot be obtained.

Inventions directed at solving the above-described problem are disclosed in Patent Literature 1 to 3. The inventions disclosed in Patent Literature 1 and 2 attempt to suppress the occurrence of false peaks by improving an optical system so as to suppress the occurrence of diffused reflected light at a place other than the examination object. The invention disclosed in Patent Literature 3 attempts to suppress the occurrence of false peaks by improving an optical system so as to change diffused reflected light that arose at a place other than the examination object into scattered light.

However, even when it is attempted to suppress the occurrence of false peaks by improving an optical system as in the inventions disclosed in the above-described literature, due to the configuration of the apparatus it is difficult to adequately suppress the occurrence of false peaks, and it is also difficult to obtain a true optical tomographic image of an examination object.

The present invention has been made to solve the above-described problems, and an object of the present invention is to provide a method for acquiring an optical tomographic image that can easily obtain a true optical tomographic image of an examination object.

### Solution to Problem

A method according to a first invention is a method for acquiring an optical tomographic image based on a result of a Fourier transform of an interference light spectrum obtained by dividing light that is output from a light source unit into first branched light and second branched light and causing mutual interference between reflected light and diffused reflected light, the reflected light arising at a reflecting body when the first branched light is irradiated onto the reflecting body and the diffused reflected light arising when the second branched light is irradiated onto an examination region, the method comprising: (1) a first step of acquiring a first optical tomographic image based on a result of a Fourier transform of the interference light spectrum when an examination object is arranged in the examination region; (2) a second step of acquiring a second optical tomographic image based on a result of a Fourier transform of the interference light spectrum when the examination object is not arranged in the examination region; and (3) a third step of acquiring an optical tomographic image of the examination object based on a difference between the first optical tomographic image and the second optical tomographic image.

A method according to a second invention is a method for acquiring an optical tomographic image based on a result of a Fourier transform of an interference light spectrum obtained by dividing light that is output from a light source unit into first branched light and second branched light and causing mutual interference between reflected light and diffused reflected light, the reflected light arising at a reflecting body when the first branched light is irradiated onto the reflecting body and the diffused reflected light arising when the second branched light is irradiated onto an examination region, the method comprising: (1) a first step of acquiring a first optical tomographic image based on a result of a Fourier transform of the interference light spectrum when an examination object is arranged in the examination region; (2) a second step of acquiring a second optical tomographic image based on a result of a Fourier transform of the interference light spectrum when a light shielding plate is arranged at a position that is nearer to the light source unit than a position at which the examination object is arranged in the examination region; and (3) a third step of acquiring an optical tomographic image of the examination object based on a difference between the first optical tomographic image and an image of an area that is farther than a position at which the light shielding plate is arranged in the second optical tomographic image.

In the method for acquiring an optical tomographic image according to the first or second invention, it is preferable that: the first step includes scanning an irradiation position of the second branched light onto the examination object and acquiring the first optical tomographic image at each irradiation position during scanning thereof; and the third step includes acquiring a two-dimensional or three-dimensional optical tomographic image of the examination object based on the first optical tomographic images at each irradiation position that are acquired in the first step and the second optical tomographic image that is acquired in the second step.

### Advantageous Effects of Invention

A method for acquiring an optical tomographic image of the present invention can easily obtain a true optical tomographic image of an examination object.

### BRIEF DESCRIPTION OF DRAWINGS

FIG. 1 is a view illustrating the configuration of an apparatus 1 for acquiring an optical tomographic image.

FIG. 2 is a view for describing a method for acquiring an optical tomographic image.

FIG. 3 is a view for describing a method for acquiring an optical tomographic image according to a first embodiment.

FIG. 4 is a view for describing a method for acquiring an optical tomographic image according to a second embodiment.

### DESCRIPTION OF EMBODIMENTS

Modes for carrying out the present invention are described in detail hereunder with reference to the accompanying drawings. Further, same elements are denoted by same reference numerals in the description of the drawings and duplicate descriptions are omitted.

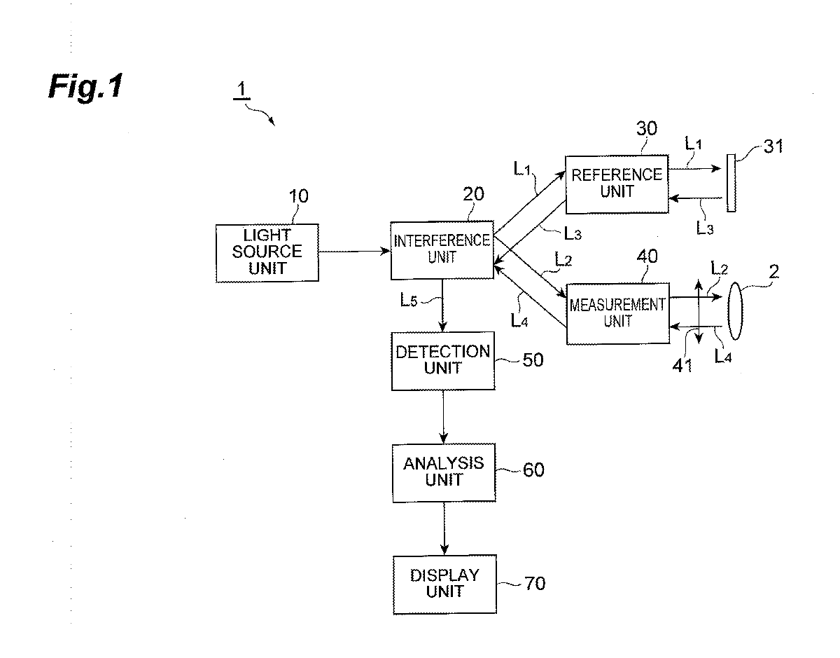

FIG. 1 is a view illustrating the configuration of an apparatus 1 for acquiring an optical tomographic image. The apparatus 1 for acquiring an optical tomographic image acquires an optical tomographic image of an examination object 2 by FD-OCT, and comprises a light source unit 10, an interference unit 20, a reference unit 30, a measurement unit 40, a detection unit 50, an analysis unit 60 and a display unit 70.

The light source unit 10 outputs light having a band. In OCT, spatial resolution in a depth direction of the examination object 2 is inversely proportional to a bandwidth of the light, and also depends on a spectrum shape. Therefore, a light source unit which can output light with a broadband spectrum and high spectral flatness is suitable as the light source unit 10. It is preferable if the light source unit 10 outputs broadband light for which the intensity is −30 dBm/nm or more over a continuous wavelength band having a bandwidth of 10 nm or more.

For example, an amplified spontaneous emission (ASE) light source that includes a rare-earth-doped glass as a light amplifying medium and that can output broadband ASE light, a supercontinuum (SC) light source that can output SC light whose band is expanded by a nonlinear optical phenomenon in an optical waveguide, or a light source including a super luminescence diode (SLD) or the like can be suitably used as the light source unit 10. Further, the light source unit 10 may be a light source unit in which the overall bandwidth becomes 10 nm as the result of temporally sweeping wavelengths, such as in a variable wavelength laser light source, or may be a light source unit in which the overall bandwidth becomes 10 nm as the result of using light of respective wavelength bands output from a plurality of light sources, respectively.

The interference unit 20 divides the light that is output from the light source unit 10 into first branched light L₁ and second branched light L₂. The interference unit 20 irradiates the first branched light L₁ onto a reflecting body 31 and receives a reflected light L₃ from the reflecting body 31 accompanying the irradiation, irradiates the second branched light L₂ onto the examination object 2 and receives a diffused reflected light L₄ from the examination object 2 accompanying the irradiation, causes the reflected light L₃ and the diffused reflected light L₄ to interfere with each other, and outputs a resulting interference light L₅ to the detection unit 50.

The reference unit 30 includes an optical system between the interference unit 20 and the reflecting body 31, and guides the first branched light L₁ from the interference unit 20 to the reflecting body 31, and guides the reflected light L₃ from the reflecting body 31 to the interference unit 20. The measurement unit 40 includes an optical system between the interference unit 20 and the examination object 2, and guides the second branched light L₂ from the interference unit 20 to the examination object 2, and guides the diffused reflected light L₄ from the examination object 2 to the interference unit 20. Further, a scanning unit 41 is provided which scans the irradiation position of the second branched light L₂ onto the examination object 2.

The detection unit 50 detects a spectrum of the interference light L₅ that is output from the interference unit 20. The analysis unit 60 subjects the interference light spectrum detected by the detection unit 50 to a Fourier transform, and acquires an optical tomographic image based on the result of the Fourier transform. By scanning the irradiation position of the second branched light L₂ onto the examination object 2 by means of the scanning unit 41, the analysis unit 60 can acquire an optical tomographic image at each irradiation position during the scanning, and can thereby acquire a two-dimensional or three-dimensional optical tomographic image. The display unit 70 displays an optical tomographic image acquired by the analysis unit 60.

A method for acquiring an optical tomographic image of the present embodiment can acquire an optical tomographic image of the examination object 2 using the apparatus 1 for acquiring an optical tomographic image having the above-described configuration.

According to the method for acquiring an optical tomographic image of the present embodiment, light that is output from the light source unit 10 is divided into the first branched light L₁ and the second branched light L₂ by the interference unit 20, and the first branched light L₁ and the second branched light L₂ are output from the interference unit 20. The first branched light L₁ that is output from the interference unit 20 is irradiated onto the reflecting body 31 via the reference unit 30. The reflected light L₃ that arises as a result of the first branched light L₁ being irradiated onto the reflecting body 31 arrives at the interference unit 20 via the reference unit 30. The second branched light L₂ that is output from the interference unit 20 is irradiated onto the examination region on which the examination object 2 is arranged, via the measurement unit 40. The diffused reflected light L₄ that arises as a result of the second branched light L₂ being irradiated onto the examination region arrives at the interference unit 20 via the measurement unit 40. The reflected light L₃ from the reference unit 30 and the diffused reflected light L₄ from the measurement unit 40 interfere with each other at the interference unit 20. A spectrum of the interference light L₅ thereof is detected by the detection unit 50. The interference light spectrum is subjected to a Fourier transform by the analysis unit 60, and an optical tomographic image is acquired based on the result of the Fourier transform.

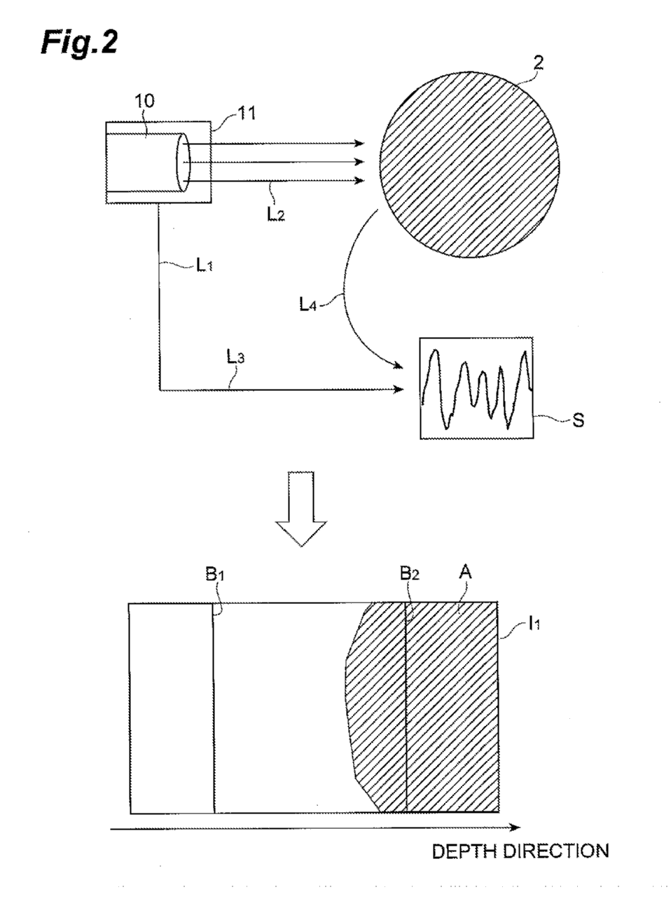

FIG. 2 is a view for describing the method for acquiring an optical tomographic image. The reflected light L₃ that arises as a result of the first branched light L₁ that is output from the interference unit 20 being irradiated onto the reflecting body 31, and the diffused reflected light L₄ that arises as a result of the second branched light L₂ that is output from the interference unit 20 being irradiated onto the examination object 2 interfere with each other at the interference unit 20. A spectrum S of the interference light L₅ that is output from the interference unit 20 is obtained by the detection unit 50, and an optical tomographic image I₁ is acquired based on the result of a Fourier transform of the interference light spectrum S by the analysis unit 60.

The optical tomographic image I₁ that is acquired at this time includes a true optical tomographic image A of the examination object 2 that is derived from the diffused reflected light that arises at the examination object 2, and false peaks B₁ and B₂ that are derived from diffused reflected light that arises at a place other than the examination object 2. The reflected light that arises at a place other than the examination object 2 includes, for example, light reflected from a light source cover 11 that is the front face of the light source unit 10, light reflected from a front end face of the light source unit 10, and light reflected from a surface or a boundary face of optical elements comprising the interference unit 20, the reference unit 30, or the measurement unit 40, and also includes light reflected by multiple reflection.

The false peak B₂ among the false peaks B₁ and B₂ that are derived from diffused reflected light that arises at a place other than the examination object 2 is superimposed on the true optical tomographic image A of the examination object 2. The method for acquiring an optical tomographic image of the present embodiment can remove the false peak B₂ from the optical tomographic image I₁ and thereby acquire the true optical tomographic image A of the examination object 2, and can also remove the false peak B₁.

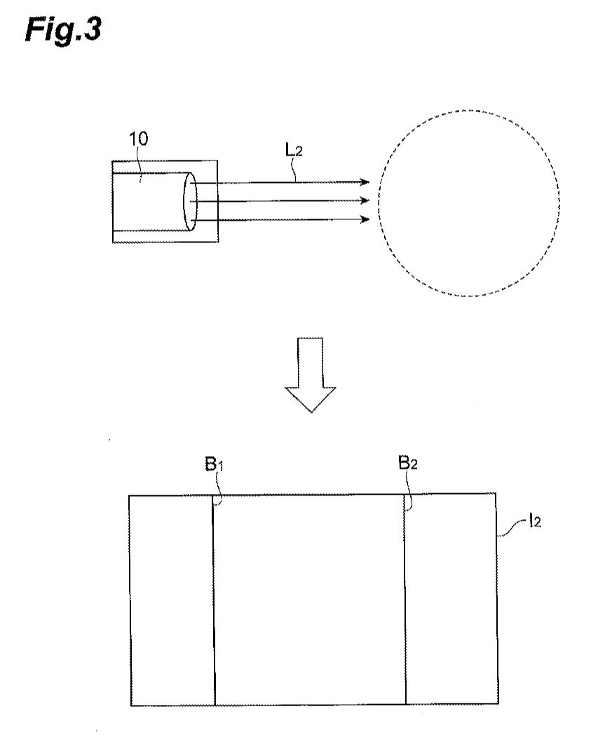

FIG. 3 is a view for describing the method for acquiring an optical tomographic image of the first embodiment. The method for acquiring an optical tomographic image of the first embodiment acquires the true optical tomographic image A of the examination object 2 through first to third steps which are described in the following. In the first step, as described above using FIG. 2, the first optical tomographic image I₁ is acquired based on the result of a Fourier transform of the interference light spectrum S when the examination object 2 is arranged in the examination region.

In the second step, a second optical tomographic image I₂ is acquired based on the result of a Fourier transform of an interference light spectrum when the examination object 2 is not arranged in the examination region. The order of executing the first step and second step is arbitrary, and measurement is performed under common conditions (excluding the point regarding the presence or absence of the examination object 2) and using a common apparatus.

Although the second optical tomographic image I₂ that is acquired in the second step does not include the true optical tomographic image A of the examination object 2 that is derived from diffused reflected light that arises at the examination object 2, the second optical tomographic image I₂ includes the false peaks B₁ and B₂ that are derived from diffused reflected light that arises at a place other than the examination object 2. Therefore, in the third step, the true optical tomographic image A of the examination object 2 is acquired based on a difference between the first optical tomographic image I₁ and the second optical tomographic image I₂. The optical tomographic image obtained in this manner does not include the false peaks B₁ and B₂.

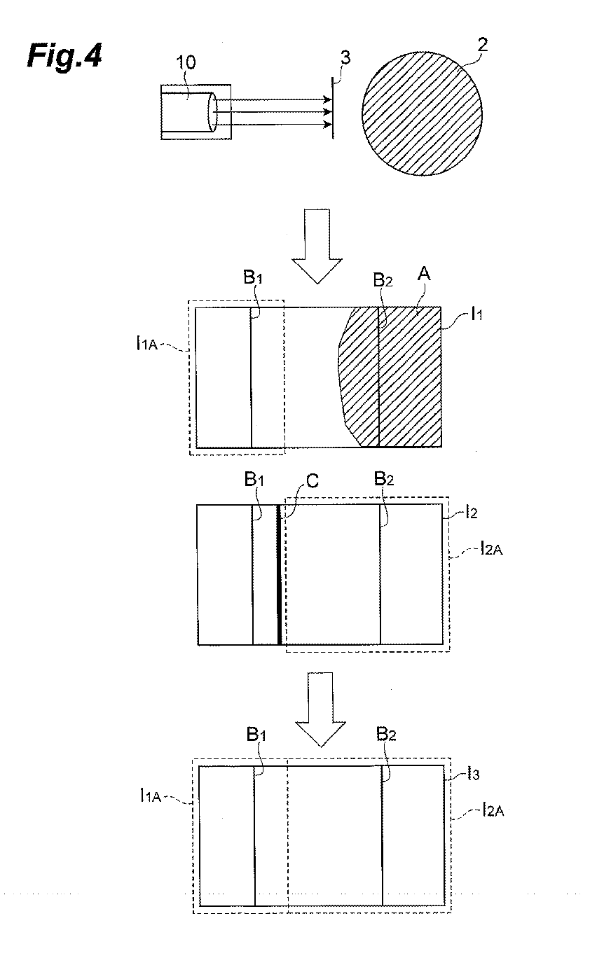

FIG. 4 is a view for describing a method for acquiring an optical tomographic image according to a second embodiment. The method for acquiring an optical tomographic image according to the second embodiment acquires the true optical tomographic image A of the examination object 2 through first to third steps that are described in the following. In the first step, as described above using FIG. 2, the first optical tomographic image I₁ is acquired based on the result of a Fourier transform of the interference light spectrum S when the examination object 2 is arranged in the examination region.

In the second step, a light shielding plate 3 is arranged at a position that is nearer to the light source unit 10 than the position at which the examination object 2 is arranged in the examination region, and the second optical tomographic image I₂ is acquired based on the result of a Fourier transform of an interference light spectrum at this time. The order of executing the first step and second step is arbitrary, and measurement is performed under common conditions (excluding the point regarding the presence or absence of the light shielding plate 3) and using a common apparatus. Further, in the second step, arrangement of the examination object 2 is arbitrary.

Although the second optical tomographic image I₂ that is acquired in the second step does not include the true optical tomographic image A of the examination object 2 that is derived from diffused reflected light that arises at the examination object 2, the second optical tomographic image I₂ includes the false peaks B₁ and B₂ that are derived from diffused reflected light that arises at a place other than the examination object 2, and also includes a peak C that is derived from diffused reflected light that arises at the light shielding plate 3.

Therefore, in the third step, the true optical tomographic image A of the examination object 2 is acquired based on a difference between the first optical tomographic image I₁ and an image I_{2A} that is an image of an area that is farther than the position at which the light shielding plate 3 is arranged in the second optical tomographic image I₂ and that includes the false peak B₂. An optical tomographic image that is obtained as a result does not include the false peak B₂. On the other hand, since the image I_{2A} does not include the false peak B₁, the false peak B₁ remains in the optical tomographic image that is obtained based on the difference. However, since the false peak B₁ is not superimposed on the true optical tomographic image A of the examination object 2, the false peak B₁ does not constitute a problem.

Alternatively, in the third step, an optical tomographic image I₃ is created by synthesizing the image I_{2A} that is an image of the area that is farther than the position at which the light shielding plate 3 is arranged in the second optical tomographic image I₂ and that includes the false peak B₂, and an image I_{1A} that is an image of an area other than the area of the image I_{2A} in the first optical tomographic image I₁ and that includes the false peak B₁. The true optical tomographic image A of the examination object 2 is then acquired based on a difference between the first optical tomographic image I₁ and the optical tomographic image I₃. This is equivalent to subtracting the image I_{2A} from the first optical tomographic image I₁, and further subtracting the image I_{1A} from the resulting image. The optical tomographic image that is obtained as a result does not include the false peaks B₁ and B₂.

Further, in each of the first and second embodiments, a configuration may be adopted in which, in the first step, the irradiation position of the second branched light L₂ onto the examination object 2 is scanned, and the first optical tomographic image I₁ is acquired at each irradiation position during the scanning, and in the third step, a two-dimensional or three-dimensional optical tomographic image A of the examination object 2 is acquired based on the first optical tomographic images I₁ at the respective irradiation positions that are acquired in the first step and the second optical tomographic image I₂ that is acquired in the second step. Scanning of the irradiation position of the second branched light L₂ is not required in the second step. Note that, two-dimensional optical tomographic images are schematically shown in FIG. 2 to FIG. 4.

As described in the foregoing, the methods for acquiring an optical tomographic image of the first and second embodiments can easily obtain the true optical tomographic image A of the examination object 2.

### INDUSTRIAL APPLICABILITY

The present invention can be applied to a method for acquiring an optical tomographic image.

### REFERENCE SIGNS LIST

1 . . . Apparatus for acquiring an optical tomographic image, 2 . . . Examination object, 3 . . . Light shielding plate, 10 . . . Light source unit, 20 . . . Interference unit, 30 . . . Reference unit, 31 . . . Reflecting body, 40 . . . Measurement unit, 41 . . . Scanning unit, 50 . . . Detection unit, 60 . . . Analysis unit, 70 . . . Display unit.

1. A method for acquiring an optical tomographic image of an examination object, the method comprising:
acquiring a first optical tomographic image including:
dividing light output from a light source unit into first branched light and second branched light;
causing mutual interference between reflected light and first diffused reflected light to form first interference light, the reflected light arising at a reflecting body irradiated with the first branched light and the first diffused reflected light arising from an examining region irradiated with the second branched light when the examining region includes the examination object; and
Fourier transforming a spectrum of the first interference light;
acquiring a second optical tomographic image including:
dividing light output from the light source unit into first branched light and second branched light;
causing mutual interference between the reflected light and second diffused reflected light to form second interference light, the second diffused reflected light arising from the examining region irradiated with the second branched light when the examining region does not include the examination object; and
Fourier transforming a spectrum of the second interference light; and
acquiring the optical tomographic image based on a difference between the first optical tomographic image and the second optical tomographic image.

2. A method for acquiring an optical tomographic image of an examination object, the method comprising:
acquiring a first optical tomographic image including:
dividing light output from a light source unit into first branched light and second branched light;
causing mutual interference between reflected light and first diffused reflected light to form first interference light, the reflected light arising at a reflecting body irradiated with the first branched light and the first diffused reflected light arising from an examining region irradiated with the second branched light when the examining region includes the examination object; and
Fourier transforming a spectrum of the first interference light;
acquiring a second optical tomographic image including:
dividing light output from the light source unit into first branched light and second branched light;
causing mutual interference between the reflected light and second diffused reflected light to form second interference light, the second diffused reflected light arising from the examining region irradiated with the second branched light when the examining region includes a light shielding plate is arranged at a position that is nearer to the light source unit than a position at which the examination object is arranged; and
Fourier transforming a spectrum of the second interference light; and
acquiring the optical tomographic image based on a difference between the first optical tomographic image and an image of an area that is farther than a position at which the light shielding plate is arranged in the second optical tomographic image.

3. The method for acquiring an optical tomographic image according to claim 1, wherein:
acquiring a first optical tomographic image includes scanning an irradiation position of the second branched light onto the examination object and acquiring the first optical tomographic image at each irradiation position during scanning thereof; and
acquiring a second optical tomographic image includes acquiring a two-dimensional or three-dimensional optical tomographic image of the examination object based on the first optical tomographic images at each irradiation position that are acquired in the first step and the second optical tomographic image that is acquired in the second step.

4. The method for acquiring an optical tomographic image according to claim 2, wherein:
acquiring a first optical tomographic image includes scanning an irradiation position of the second branched light onto the examination object and acquiring the first optical tomographic image at each irradiation position during scanning thereof; and
acquiring a second optical tomographic image includes acquiring a two-dimensional or three-dimensional optical tomographic image of the examination object based on the first optical tomographic images at each irradiation position that are acquired in the first step and the second optical tomographic image that is acquired in the second step.
